(19) United States
(12) Patent Application Publication
(10) Pub. No.: US 2006/0154981 A1
(43) Pub. Date: Jul. 13, 2006

(54) Method of reducing intraocular pressure and treating glaucoma

(75) Inventors: Peter G. Klimko, Fort Worth, TX (US); Mark R. Hellberg, Arlington, TX (US); Wan-Heng Wang, Grapevine, TX (US); Abbot F. Clark, Arlington, TX (US); Loretta Graves McNatt, Hurst, TX (US)

Correspondence Address: Alcon Research, Ltd.;IP Legal Department, 6201 So. Freeway, Fort Worth, TX, 76134-2099, US

(73) Assignee: Alcon, Inc.

(21) Appl. No.: 11/330,634

(22) Filed: Jan. 12, 2006

Related U.S. Application Data
(60) Provisional application No. 60/643,284, filed on Jan. 12, 2005.

(51) Int. Cl. A61K 31/366; A61K 31/22; A61K 31/19

(52) U.S. Cl. 514/460; 514/546; 514/557

(57) ABSTRACT

The use of 5,6,7-trihydroxyheptanoic acid and analogs for the treatment of ocular hypertension and glaucoma is disclosed.

This application claims priority to U.S. Provisional Application, U.S. Ser. No. 60/643,284 filed Jan. 12, 2005.

The present invention is directed to the treatment of ocular disorders. In particular, the present invention is directed toward the use of 5,6,7-trihydroxyheptanoic acid and its analogs to treat glaucoma and ocular hypertension in mammals.

### BACKGROUND OF THE INVENTION

Glaucoma is an ocular disease that is usually associated with elevated intraocular pressure that causes damage to the optic nerve and vision loss, though it can also be present in patients who have normal eye pressure measurements. Glaucoma can usually be controlled with prescription eye drops. In some cases, treatment with lasers or surgery to relieve intraocular pressure may be required.

Ocular hypertension (elevated intraocular pressure) is commonly treated using therapeutic agents that suppress aqueous humor production. These agents include beta-blockers, such as timolol and betaxolol, and carbonic anhydrase inhibitors, such as dorzolamide and brinzolamide. Recently, the use of prostaglandin analogs, which are believed to reduce intraocular pressure by increasing uveoscleral outflow, has become common. Three marketed prostaglandin analogs are latanoprost, bimatoprost and travoprost.

It is widely believed that ocular hypertension and associated primary open angle glaucoma are fundamentally a disease of the trabecular meshwork and are associated with increased extracellular matrix deposition that is caused by an imbalance between extracellular matrix formation and removal.

Lee et. al. have disclosed that compounds 1 (R═H) and 2 inhibit LTB₄-induced chemotaxis of neutrophils as potently as lipoxin A₄ [Lee et. al., Biochemical and Biophysical Research Communications 1991, 180 (3), 1416-21]. A definitive experiment of trying to reverse the chemotaxis inhibition seen with 1 and 2 by for example using a receptor antibody or small molecule antagonist of the receptor was not performed however, so it is unclear whether the bioeffects of compounds 1 and 2 in this system are due to activation of the lipoxin A₄ receptor. Lipoxin A₄ and certain analogs thereof have been reported to be anti-inflammatory agents [see for example Serhan et. al., U.S. Pat. No. 5,441,951 for compound 3 (R═H); Levy et al., Nature Medicine 2002, 8, 1018-1023, for compound 4 (R═H); Guilford et al., Journal of Medicinal Chemistry 2004, 47, 2157-2165 for compound 5 (R═H)]. Certain lipoxin analogs have been disclosed for treating dry eye (Gamache et al., U.S. Pat. No. 6,645,978 B1) and for treating glaucoma (Bauman et al., U.S. Pat. No. 6,831,186 B2), and the methyl ester of lipoxin A₄ has been reported to lower IOP in rabbits [Cotran, P. R., Hsu, C.; Serhan, C. N. Abstract of paper presented at the 1995 meeting of the Association for Research in Vision and Ophthalmology, abstract number 3812; Investigative Ophthalmology and Visual Science 1995, 36 (4), 3812]. However to the best of our knowledge no compounds of the present invention have been described for treating ocular hypertension or glaucoma.

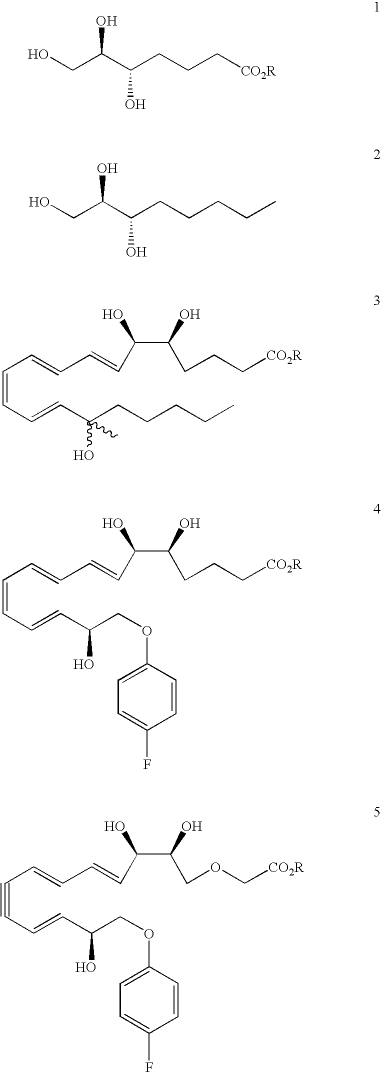

### SUMMARY OF THE INVENTION

The present invention is directed to methods for the treatment of glaucoma and ocular hypertension. According to the methods of the present invention, a 5,6,7-trihydroxyheptanoic acid or analog is administered to a patient suffering from glaucoma or ocular hypertension. The 5,6,7-trihydroxyheptanoic acid or analog is preferably administered in an ophthalmic composition dosed topically to a patient's eye.

Without intending to be bound by any theory, it is believed that the compounds of the present invention treat ocular hypertension and glaucoma by inhibiting and reversing inappropriate extracellular matrix buildup in the trabecular meshwork.

### DETAILED DESCRIPTION OF THE INVENTION

Unless indicated otherwise, all component amounts are presented on a % (w/v) basis.

According to the methods of the present invention, a composition comprising a compound of formula I is administered to a mammal suffering from or at risk of suffering from ocular hypertension or glaucoma but not suffering from dry eye or uveitis. 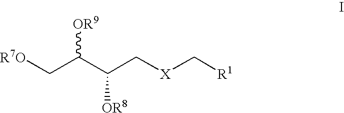
wherein
R¹ is C₂H₅, CO₂R, CO₂⁻M⁺, CONR²R³, CH₂OR⁴, or CH₂NR⁵R⁶;
R is H, C₁-C₆ straight chain or branched alkyl, C₃-C₆ straight chain or branched alkenyl, C₃-C₆ straight chain or branched alkynyl, C₃-C₆ cycloalkyl, benzyl, or phenyl;
M⁺ is Li⁺, Na⁺, K⁺, or an ammonium moiety of formula ⁺NR¹⁰R¹¹R¹² R¹³, where R¹⁰-R¹³ are independently H or C₁₋₆ alkyl, each alkyl group optionally bearing an OH or OCH₃ substituent;
R², R³ are independently H, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
R⁴ is H, C₁₋₆ alkyl, C₃₋₆ cycloalkyl, benzyl, phenyl, or C(O)R¹⁴;
R¹⁴ is H, C₁₋₆ alkyl, C₃₋₆ cycloalkyl, benzyl, phenyl, or OR¹⁵;
R¹⁵ is C₁₋₆ alkyl, C₃₋₆ cycloalkyl, benzyl, or phenyl;
R⁵, R⁶ are independently H, C(O)R¹⁴, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
X is CH₂, O, or S;
R⁷, R⁸, and R⁹ are independently H, CH₃, C₂H₅, C(O)R¹⁴, CO₂R¹⁵, CONH₂, or CONHR¹⁵;
or R⁷ and R⁹ or R⁸ and R⁹ together constitute a carbonyl group (C═O), thus forming a cyclic carbonate;
or OR⁸R¹ together form a cyclic ester; and
[symbol] indicates that the substituents at the OR⁹-bearing carbon can be arranged to afford either the R or S absolute configuration:

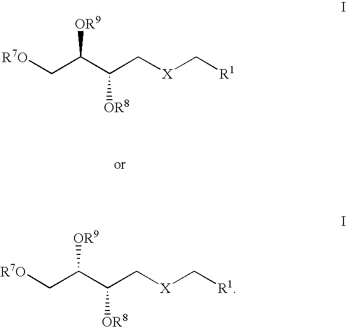

Preferred compounds of formula I are those wherein:
R¹ is C₂H₅, CO₂R, or CO₂⁻M⁺;
R is H, CH₃, C₂H₅, n-C₃H₇, or i-C₃H₇;
M⁺ is Li⁺, Na⁺, K⁺, or NH₄⁺;
X is CH₂; and
R⁷, R⁸, and R⁹ are independently H or C(O)CH₃; or
R⁷ and R⁸ or R⁸ and R⁹ together constitute a carbonyl group (C═O), thus forming a cyclic carbonate; or
OR⁸R¹ together form a cyclic ester (a lactone).

Among the especially preferred are compounds 1, 2, and 6-9. Compound 1 is commercially available from Biomol Research Laboratories, Plymouth Meeting, Pa., and compound 2 can be prepared as detailed in Lee et. al., Biochemical and Biophysical Research Communications 1991, 180 (3), 1416-21. Compounds 6-9 can be prepared as described in examples 1-4 below.

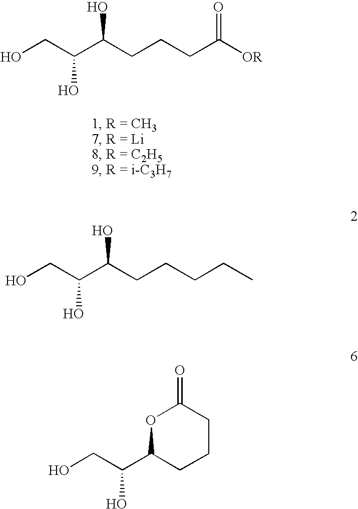

### EXAMPLE 1

### Synthesis of Compound 6

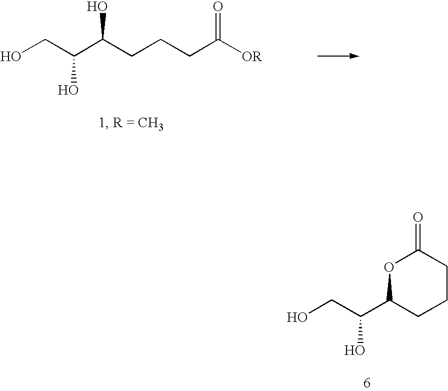

A solution of methyl ester 1 (20 mg, 0.104 mmol) in MeOH (2.1 mL) containing 1 M LiOH (0.5 mL, 0.5 mmol) was heated in a microwave heater at 120° C. for 6 minutes. The reaction was concentrated and the residue was chromatographed on a 10 mm diameter×18 cm tall C18 reverse-phase silica gel column eluting with 7:3 v:v 0.05 M HCl:acetonitrile to afford a crude white solid after concentration (40.9 mg). The solid was rinsed with hot CH₃CN (2×2 mL) and the filtrate was concentrated to afford lactone 6 (7.8 mg, 47%). ¹³C NMR (150 MHz, dmso-d₆) δ 171.12 (C), 79.86 (CH), 72.44 (CH), 62.03 (CH₂), 29.39 (CH₂), 21.67 (CH₂), 17.55 (CH₂).

### EXAMPLE 2

### Synthesis of Compound 7

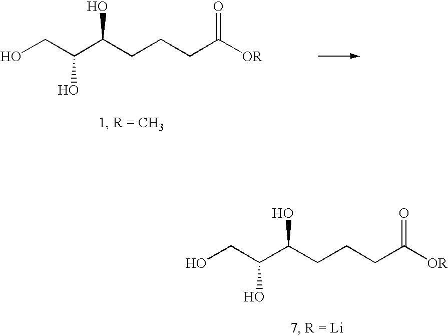

A solution of methyl ester 1 in aqueous MeOH is heated to reflux in the presence of 3 equivalents of lithium hydroxide. After 6 h the reaction is cooled to room temperature and the pH of the solution is adjusted to 6 by the addition of 70-9 mesh sulfonic acid resin MP (commercially available from Novabiochem/EMD Biosciences, 10394 Pacific Center Court, San Diego, Calif. 92121). The solution is filtered through a 0.2 μM poly-terfluoroethylene syringe filter and concentrated to afford the lithium carboxylate 7 as a white solid. ¹H NMR (D₂O, 400 MHz) 63.69-3.64 (m, 1H), 3.55-3.47 (m, 3H), 2.16-2.12 (m, 2H), 1.67-1.64 (m, 1H), 1.54-1.48 (m, 2H), 1.38-1.34 (m, 1H). ¹³C NMR (D₂O, 100 MHz) δ 183.46 (C), 74.61 (CH), 71.67 (CH), 62.49 (CH₂), 37.26 (CH₂), 31.55 (CH₂), 22.04 (CH₂).

### EXAMPLE 3

### Synthesis of Compound 8

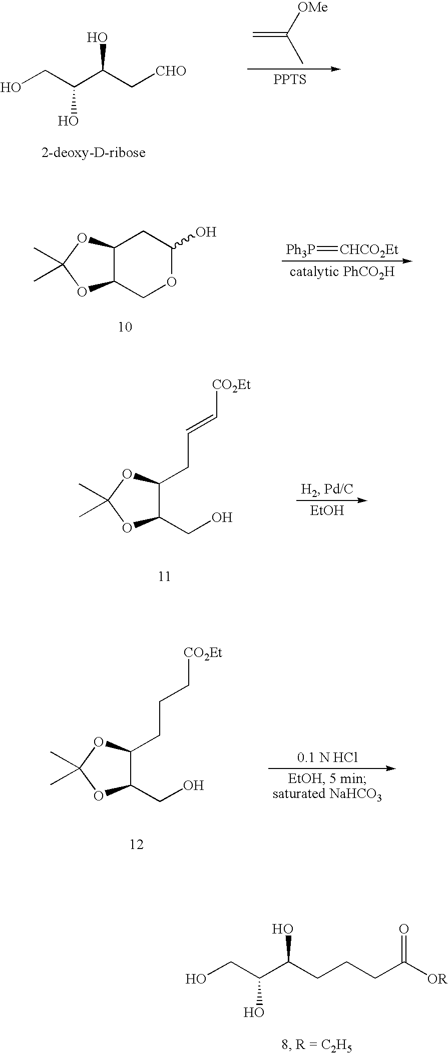

2-deoxy-D-ribose is converted to the acetonide-protected lactol 10 by treatment with 2-methoxypropene and catalytic pyridinium p-toluenesulfonate (PPTS) in ethyl acetate. Wittig reaction with Ph₃P═CHCO₂Et in THF in the presence of catalytic benzoic acid affords enoate 11, which is reduced to 12 under a hydrogen atmosphere in the presence of catalytic Pd/C in ethanol. Deprotection of 12 using 0.1 N HCl in ethanol for 5 minutes, followed by quenching with aqueous NaHCO₃, affords 8 after silica gel chromatographic purification.

### EXAMPLE 4

### Synthesis of Compound 9

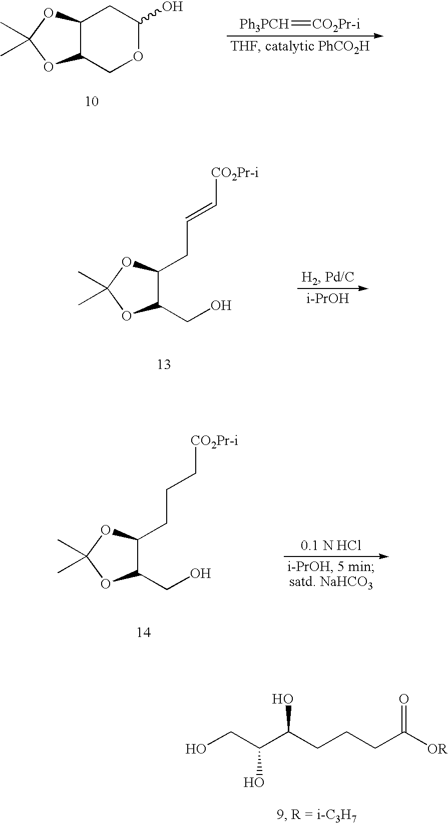

Wittig reaction of lactol 10 with Ph₃P═CHCO₂Et in THF in the presence of catalytic benzoic acid affords enoate 13, which is reduced to 14 under a hydrogen atmosphere in the presence of catalytic Pd/C in isopropanol. Deprotection of 14 using 0.1 N HCl in isopropanol for 5 minutes, followed by quenching with aqueous NaHCO₃, affords 9 after silica gel chromatographic purification.

According to the methods of the present invention, a compound of formula I is administered in a pharmaceutically acceptable carrier or implant. Preferably, the compound of formula I is administered topically in the form of an eye drop. The compositions and implants are formulated in accordance with methods known in the art. The compositions and implants may contain more than one compound of formula I. Additionally, the compositions and implants may contain a second drug, other than a compound of formula I.

The compositions and implants used in the methods of the present invention contain a pharmaceutically effective amount of a compound of formula I. Generally, the topically administrable compositions used in the methods of the present invention will contain from 0.001 to 2% of a compound of formula I. Preferably, the compositions of the present invention will contain from 0.01 to 1% of a compound of formula I.

The topical compositions administered according to the present invention may also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 5.5-8.

Topical ophthalmic products are typically packaged in multidose form. Preservatives are typically required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically will not contain a preservative and will be unpreserved.

Generally, 1-2 drops of topical compositions containing a compound of formula I will be administered from 1-3 times per day.

A representative eye drop formulation is provided below in Example 5.

### EXAMPLE 5

| Ingredient | Concentration (% w/v) |
|---|---|
| Compound of formula I | 0.001-2 |
| Benzododecinium Bromide | 0.01-0.015 |
| Boric Acid | 0.2-0.4 |
| Xanthan Gum | 0.5-0.7 |
| Edetate Disodium | 0-0.01 |
| Polysorbate 80 | 0.05 |
| Mannitol | q.s. to 250-300 mOsm. |
| NaOH/HCl | q.s. to pH 6-8 |
| Purified Water | q.s. to 100% |

### EXAMPLE 6

The ability of compound 1 of the present invention to reduce intraocular pressure (IOP) was evaluated in cynomolgus monkeys with ocular hypertension produced by previous laser trabeculoplasty in the right eye. Animals had been grained to sit in restraint chairs and conditioned to accept experimental procedures without chemical restraint. Animals were administered a 30 μL drop of 300 μg of compound 1 dissolved in vehicle to the lasered eye. IOP was determined with a pneumatonometer after light corneal anesthesia with dilute proparacaine. Baseline IOP was measured at 0 hours, and drug was dosed 35 minutes later.

| Time | Mean IOP, | Change in IOP, | % Change |
|---|---|---|---|
| (hour) | mm Hg | mm Hg | in IOP |
| 0 | 39.5 | 0.0 | 0.0 |
| 1 | 36.8 | −2.8 | −6.9 |
| 3 | 33.4 | −6.1 | −15.6 |
| 6 | 33.9 | −5.6 | −14.1 |

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

What is claimed is:

1. A method for the treatment of ocular hypertension or glaucoma in a mammal not suffering from dry eye or uveitis, which comprises administering to the mammal a pharmaceutically effective amount of a compound of formula I: 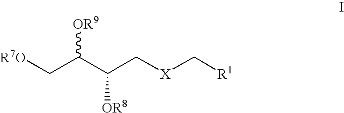
wherein
R¹ is C₂H₅, CO₂R, CO₂⁻M⁺, CONR²R³, CH₂OR⁴, or CH₂NR⁵R⁶;
R is H, C₁-C₆ straight chain or branched alkyl, C₃-C₆ straight chain or branched alkenyl, C₃-C₆ straight chain or branched alkynyl, C₃-C₆ cycloalkyl, benzyl, or phenyl;
M⁺ is Li⁺, Na⁺, K⁺, or an ammonium moiety of formula ⁺NR¹⁰R¹¹R¹²R¹³, where R¹⁰-R¹³ are independently H or C₁₋₆ alkyl, each alkyl group optionally bearing an OH or OCH₃ substituent;
R², R³ are independently H, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
R⁴ is H, C₁₋₆ alkyl, C₃₋₆ cycloalkyl, benzyl, phenyl, or C(O)R¹⁴;
R¹⁴ is H, C₁₋₆ alkyl, C₃₋₆ cycloalkyl, benzyl, phenyl, or OR¹⁵;
R¹⁵ is C₁₋₆ alkyl, C₃₋₆ cycloalkyl, benzyl, or phenyl;
R⁵, R⁶ are independently H, C(O)R¹⁴, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
X is CH₂, O, or S;
R⁷, R⁸, and R⁹ are independently H, CH₃, C₂H₅, C(O)R¹⁴, CO₂R¹⁵, CONH₂, or CONHR¹⁵;
or R⁷ and R⁹ or R⁸ and R⁹ together constitute a carbonyl group (C═O), thus forming a cyclic carbonate;
or OR⁸R¹ together form a cyclic ester; and [symbol]
indicates that the substituents at the OR⁹-bearing carbon can be arranged to afford either the R or S absolute configuration:

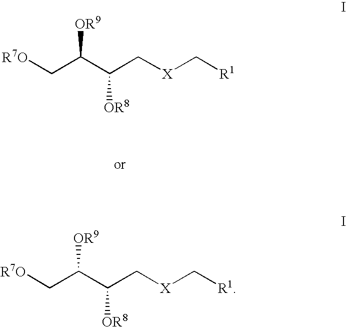

2. The method of claim 1 wherein for the compound of formula I:
R¹ is C₂H₅, CO₂R, or CO₂⁻M⁺;
R is H, CH₃, C₂H₅, n-C₃H₇, or i-C₃H₇;
M⁺ is Li⁺, Na⁺, K⁺, or NH₄⁺;
X is CH₂; and
R⁷, R⁸, and R⁹ are independently H or C(O)CH₃; or
R⁷ and R⁸ or R⁸ and R⁹ together constitute a carbonyl group (C═O), thus forming a cyclic carbonate; or
OR⁸R¹ together form a cyclic ester (a lactone).

3. The method of claim 2 wherein the compound of formula I is selected from the group consisting of:

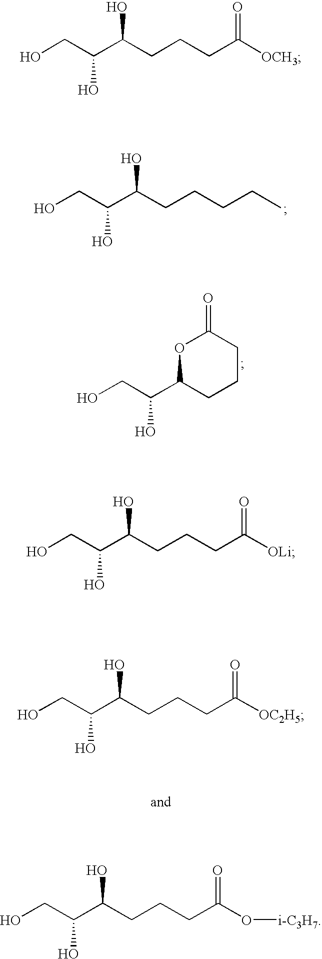

4. The method of claim 1 wherein the compound of formula I is administered in an implant.

5. The method of claim 1 wherein the compound of formula I is administered topically to the eye in a composition comprising a pharmaceutically acceptable carrier.

6. The method of claim 5 wherein the pharmaceutically effective amount of compound of formula I in the composition is from 0.001 to 2% (w/v).

7. The method of claim 6 wherein the pharmaceutically effective amount is from 0.01 to 1% (w/v).

8. The method of claim 5, wherein the pharmaceutically acceptable carrier comprises one or more ingredients selected from the group consisting of surfactants; tonicity agents; buffers; preservatives; co-solvents; and viscosity building agents.
